(19) United States
(12) Patent Application Publication
(10) Pub. No.: US 2008/0140450 A1
(43) Pub. Date: Jun. 12, 2008

(54) Treatment of metabolic syndrome with norfluoxetine

(75) Inventor: James R. Hauske, La Jolla, CA (US)

Correspondence Address: ROPES & GRAY LLP, PATENT DOCKETING 39/41, ONE INTERNATIONAL PLACE, BOSTON, MA, 02110-2624, US

(73) Assignee: AMPLA Pharmaceuticals Inc., La Jolla, CA (US)

(21) Appl. No.: 11/998,167

(22) Filed: Nov. 28, 2007

Related U.S. Application Data
(60) Provisional application No. 60/861,581, filed on Nov. 28, 2006.
(60) Provisional application No. 60/963,741, filed on Aug. 6, 2007.
(60) Provisional application No. 60/967,352, filed on Sep. 4, 2007.

(51) Int. Cl. G06Q 50/00; A61K 31/135; A61K 31/40; A61K 31/351; A61K 31/44; A61K 31/4164; A61P 3/00

(52) U.S. Cl. 705/2; 514/651; 514/423; 514/460; 514/356; 514/401; 514/402

(57) ABSTRACT

The invention relates to methods of treating metabolic syndrome, or the specific disorders associated with metabolic syndrome, comprising the administration of norfluoxetine enriched for the (R) or (S) enantiomer conjointly with a statin, a calcium channel blocker, halofenate, captopril, or an imidazoline receptor agonist (e.g., an I₁, I₂, or I₃ receptor agonist).

### RELATED APPLICATIONS

This application claims the benefit of priority to U.S. Provisional Patent Application No. 60/861,581, filed Nov. 28, 2006, U.S. Provisional Patent Application No. 60/963,741, filed Aug. 6, 2007, and U.S. Provisional Patent Application No. 60/967,352, filed Sep. 4, 2007, which applications are hereby incorporated by reference in their entirety.

### BACKGROUND

### Metabolic Syndrome

Metabolic syndrome (also known as “syndrome X,” “dysmetabolic syndrome,” “obesity syndrome,” and “Reaven's syndrome”) has emerged as a growing problem. For example, metabolic syndrome has become increasingly common in the United States. It is estimated that about 47 million adults in the United States have the syndrome.

Metabolic syndrome is generally a constellation of metabolic disorders that all result from, or are associated with, a primary disorder of insulin resistance. Accordingly, the syndrome is sometimes referred to as “insulin resistance syndrome.” Insulin resistance is characterized by disorders in which the body cannot use insulin efficiently and the body's tissues do not respond normally to insulin. As a result, insulin levels become elevated in the body's attempt to overcome the resistance to insulin. The elevated insulin levels lead, directly or indirectly, to the other metabolic abnormalities.

Some people are genetically predisposed to insulin resistance, while other people acquire factors that lead to insulin resistance. Acquired factors, such as excess body fat and physical inactivity, can elicit insulin resistance, and more broadly, clinical metabolic syndrome. Because of this relationship between insulin resistance and metabolic syndrome, it is believed that the underlying causes of this syndrome are obesity, physical inactivity and genetic factors. In fact, most people with insulin resistance and metabolic syndrome have central obesity (excessive fat tissue in and around the abdomen). The biologic mechanisms at the molecular level between insulin resistance and metabolic risk factors are not yet fully understood and appear to be complex.

Metabolic syndrome is typically characterized by a group of metabolic risk factors that include 1) central obesity; 2) atherogenic dyslipidemia (blood fat disorders comprising mainly high triglycerides (“TG”) and low HDL-cholesterol (interchangeably referred to herein as “HDL”) that foster plaque buildups in artery walls); 3) raised blood pressure; 4) insulin resistance or glucose intolerance (the body can't properly use insulin or blood sugar); 5) prothrombotic state (e.g., high fibrinogen or plasminogen activator inhibitor in the blood); and 6) a proinflammatory state (e.g., elevated high-sensitivity C-reactive protein in the blood). The National Cholesterol Education Program (NCEP) Adult Treatment Panel (ATP) III guidelines define metabolic syndrome by the following five clinical parameters: a) a waist circumference greater than 102 cm for men, and greater than 88 cm for women; b) a triglyceride level greater than 150 mg/dl; c) an HDL-cholesterol less than 40 mg/dl for men, and less than 50 mg/dl for women; d) a blood pressure greater than or equal to 130/85 mmHG; and e) a fasting glucose greater than 110 mg/dl.

According to the American Heart Association, however, there are no well-accepted criteria for diagnosing metabolic syndrome. Some guidelines suggest that metabolic syndrome involves four general factors: obesity; diabetes; hypertension; and high lipids. According to the NCEP ATP III guidelines above, the presence of at least three of these five factors meets the medical diagnosis of metabolic syndrome.

Although there is no complete agreement on the individual risk or prevalence of each factor, it is known that the syndrome, as generally agreed upon by those skilled in the field, poses a significant health risk to individuals. A person having one factor associated with the syndrome has an increased risk for having one or more of the others. The more factors that are present, the greater the risks to the person's health. When the factors are present as a group, i.e., metabolic syndrome, the risk for cardiovascular disease and premature death is very high.

For example, a person with the metabolic syndrome is at an increased risk of coronary heart disease, other diseases related to plaque buildups in artery walls (e.g., stroke and peripheral vascular disease), and type 2 diabetes. It is also known that when diabetes occurs, the high risk of cardiovascular complications increases.

Generally, patients suffering from the syndrome are prescribed a change in lifestyle, i.e., an increase in exercise and a change to a healthy diet. The goal of exercise and diet programs is to reduce body weight to within 20% of the “ideal” body weight calculated for age and height.

In some cases, diet and exercise regimens are supplemented with treatments for lipid abnormalities, clotting disorders, and hypertension. For example, patients with the syndrome typically have several disorders of coagulation that make it easier to form blood clots within blood vessels. These blood clots are often a precipitating factor in developing heart attacks. Patients with the syndrome are often placed on daily aspirin therapy to specifically help prevent such clotting events. Furthermore, high blood pressure is present in more than half the people with the syndrome, and in the setting of insulin resistance, high blood pressure is especially important as a risk factor. Some studies have suggested that successfully treating hypertension in patients with diabetes can reduce the risk of death and heart disease by a substantial amount. Additionally, patients have been treated to specifically reduce LDL-cholesterol (interchangeably referred to herein as “LDL”) levels, reduce triglyceride levels, and raise HDL levels. Given the increasing prevalence of this syndrome, there remains a need for additional and effective treatments of the syndrome.

### SUMMARY OF INVENTION

Fluoxetine is a racemate of two enantiomeric forms. The biological and pharmacological activity of each enantiomer has been found to be essentially the same; see, Robertson et al., J. Med. Chem., 31, 1412 (1988) and references cited therein. Norfluoxetine [3-(4-trifluoromethylphenoxy)-3-phenylpropylamine] is a metabolite of fluoxetine and is known to block monoamine uptake, especially serotonin. See U.S. Pat. No. 4,313,896. Since norfluoxetine it is a metabolite of fluoxetine, it is believed that this compound contributes in part to the biological activity seen upon administration of fluoxetine.

The present invention provides a method of treating or preventing metabolic syndrome in a mammal comprising administering a compound of the present invention (e.g., (R)- or (S)-norfluoxetine) or a salt or solvate thereof conjointly with a statin or a calcium channel blocker.

The present invention provides a method of treating or preventing a disorder associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with a statin or a calcium channel blocker.

The present invention provides a method of treating or preventing metabolic syndrome in a mammal comprising administering a compound of the present invention (e.g., (R)- or (S)-norfluoxetine) or a salt or solvate thereof conjointly with halofenate.

The present invention provides a method of treating or preventing the specific disorders associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with halofenate.

The present invention provides a method of treating or preventing metabolic syndrome in a mammal comprising administering a compound of the present invention (e.g., (R)- or (S)-norfluoxetine) or a salt or solvate thereof conjointly with captopril.

The present invention provides a method of treating or preventing a disorder associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with captopril.

The present invention provides a method of treating or preventing metabolic syndrome in a mammal comprising administering a compound of the present invention (e.g., (R)- or (S)-norfluoxetine) or a salt or solvate thereof conjointly with an imidazoline receptor agonist (e.g., an I₁, I₂, or I₃ receptor agonist).

The present invention provides a method of treating or preventing a disorder associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with an imidazoline receptor agonist (e.g., an I₁, I₂, or I₃ receptor agonist).

In preferred embodiments of the present invention, the mammal being treated is a human.

In certain embodiments of the present invention, norfluoxetine is enriched for either the (R) or the (S) enantiomer. In some embodiments of the present invention, norfluoxetine is enriched for the (R) enantiomer. In some embodiments of the present invention, (R)-norfluoxetine is substantially free of the (S) enantiomer. In some embodiments of the present invention, norfluoxetine is enriched for the (S) enantiomer. In some embodiments of the present invention, (S)-norfluoxetine is substantially free of the (R) enantiomer.

In certain embodiments, the present invention provides a kit comprising a first pharmaceutical formulation comprising a compound of the present invention (e.g., norfluoxetine enriched for the (R) or the (S) enantiomer); a second pharmaceutical formulation comprising at least one of the following: a statin, a calcium channel blocker, halofenate, captopril, or an imidazoline receptor agonist (e.g., an I₁, I₂, or I₃ receptor agonist); and instructions for the administration of the first and second pharmaceutical formulations.

In certain embodiments, the invention relates to a method for conducting a pharmaceutical business, by manufacturing a formulation or kit as described herein, and marketing to healthcare providers the benefits of using the formulation or kit in the treatment or prevention of metabolic syndrome or the specific disorders associated with metabolic syndrome.

In certain embodiments, the invention relates to a method for conducting a pharmaceutical business, by providing a distribution network for selling a formulation or kit as described herein, and providing instruction material to patients or physicians for using the formulation to treat or prevent metabolic syndrome or the specific disorders associated with metabolic syndrome.

In certain embodiments, the invention comprises a method for conducting a pharmaceutical business, by determining an appropriate formulation and dosage of a compound of the present invention (e.g., norfluoxetine enriched for the (R) or the (S) enantiomer) to be administered conjointly with a statin, a calcium channel blocker, halofenate, captopril, or an imidazoline receptor agonist (e.g., an I₁, I₂, or I₃ receptor agonist) in the treatment or prevention of metabolic syndrome or the specific disorders associated with metabolic syndrome, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of a compound of the present invention (e.g., norfluoxetine enriched for the (R) or the (S) enantiomer) to be administered conjointly with a statin, a calcium channel blocker, halofenate, captopril, or an imidazoline receptor agonist (e.g., an I₁, I₂, or I₃ receptor agonist) in the treatment or prevention of metabolic syndrome or the specific disorders associated with metabolic syndrome, and licensing, to a third party, the rights for further development and sale of the formulation.

### DETAILED DESCRIPTION OF THE INVENTION

The present invention relates to methods of treatment with norfluoxetine. In certain embodiments, the therapeutic preparation may be enriched to provide predominantly one enantiomer of norfluoxetine. An enantiomerically enriched mixture may comprise, for example, at least 60 mol percent of one enantiomer, or more preferably at least 75, 90, 95, or even 99 mol percent. In certain embodiments, norfluoxetine is enriched in the (R) enantiomer. In certain embodiments, (R)-norfluoxetine is substantially free of the (S)-enantiomer. In certain embodiments, norfluoxetine is enriched in the (S) enantiomer. In certain embodiments, (S)-norfluoxetine is substantially free of the (R)-enantiomer. Substantially free, as the term is used herein, means that the contaminant or less desired substance makes up less than 10%, or less than 5%, or less than 4%, or less than 3%, or less than 2%, or less than 1% as compared to the amount of the compound of interest, e.g., in the composition or compound mixture. For example, if a composition or compound mixture contains 98 grams of the (R)-enantiomer and 2 grams of the (S)-enantiomer, it would be said to contain 98 mol percent of the (R)-enantiomer and only 2% of the (S)-enantiomer.

The present invention provides a method of treating or preventing metabolic syndrome in a mammal comprising administering a compound of the present invention (e.g., (R)- or (S)-norfluoxetine) or a salt or solvate thereof conjointly with a statin or a calcium channel blocker.

The present invention provides a method of treating or preventing the specific disorders associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with a statin or a calcium channel blocker.

In certain embodiments of methods of the invention wherein a compound of the present invention (e.g., (R)- or (S)-norfluoxetine) or a salt or solvate thereof is administered to a mammal conjointly with a statin or a calcium channel blocker for the treatment of obesity, the mammal is in need of anti-psychotic treatment. In certain embodiments of methods of the invention wherein a compound of the present invention (e.g., (R)- or (S)-norfluoxetine) or a salt or solvate thereof is administered to a mammal conjointly with a statin or a calcium channel blocker for the treatment of obesity, the mammal is being treated with one or more anti-psychotic agents.

The present invention provides a method of treating or preventing metabolic syndrome in a mammal comprising administering a compound of the present invention (e.g., (R)- or (S)-norfluoxetine) or a salt or solvate thereof conjointly with halofenate or a pharmaceutically acceptable salt thereof.

The present invention provides a method of treating or preventing the specific disorders associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with halofenate or a pharmaceutically acceptable salt thereof.

In certain embodiments of methods of the invention wherein a compound of the present invention (e.g., (R)- or (S)-norfluoxetine) or a salt or solvate thereof is administered to a mammal conjointly with halofenate for the treatment of obesity, the mammal is in need of anti-psychotic treatment. In certain embodiments of methods of the invention wherein a compound of the present invention (e.g., (R)- or (S)-norfluoxetine) or a salt or solvate thereof is administered to a mammal conjointly with halofenate for the treatment of obesity, the mammal is being treated with one or more anti-psychotic agents.

In certain embodiments, wherein norfluoxetine enriched for the (R) or (S) enantiomer is administered conjointly with halofenate for methods of the invention, halofenate is enriched for the (R) or (S) enantiomer or a pharmaceutically acceptable salt thereof. An enantiomerically enriched mixture may comprise, for example, at least 60 mol percent of one enantiomer, or more preferably at least 75, 90, 95, or even 99 mol percent. In certain embodiments, halofenate is enriched in the (R) enantiomer. In certain embodiments, (R)-halofenate is substantially free of the (S)-enantiomer. In certain embodiments, halofenate is enriched in the (S) enantiomer. In certain embodiments, (S)-halofenate is substantially free of the (R)-enantiomer.

The present invention provides a method of treating or preventing metabolic syndrome in a mammal comprising administering a compound of the present invention (e.g., (R)- or (S)-norfluoxetine) or a salt or solvate thereof conjointly with captopril or a pharmaceutically acceptable salt thereof.

The present invention provides a method of treating or preventing the specific disorders associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with captopril or a pharmaceutically acceptable salt thereof.

In certain embodiments of methods of the invention wherein a compound of the present invention (e.g., (R)- or (S)-norfluoxetine) or a salt or solvate thereof is administered to a mammal conjointly with captopril for the treatment of obesity, the mammal is in need of anti-psychotic treatment. In certain embodiments of methods of the invention wherein a compound of the present invention (e.g., (R)- or (S)-norfluoxetine) or a salt or solvate thereof is administered to a mammal conjointly with captopril for the treatment of obesity, the mammal is being treated with one or more anti-psychotic agents.

The present invention provides a method of treating or preventing metabolic syndrome in a mammal comprising administering a compound of the present invention (e.g., (R)- or (S)-norfluoxetine) or a salt or solvate thereof conjointly with an imidazoline receptor agonist (e.g., an I₁, I₂, or I₃ receptor agonist).

The present invention provides a method of treating or preventing the specific disorders associated with metabolic syndrome, such as obesity, diabetes, hypertension, and hyperlipidemia, in a mammal comprising administering a compound of the present invention (e.g., (R)- or (S)-norfluoxetine) or a salt or solvate thereof conjointly with an imidazoline receptor agonist (e.g., an I₁, I₂, or I₃ receptor agonist).

In certain embodiments of methods of the invention wherein a compound of the present invention (e.g., (R)- or (S)-norfluoxetine) or a salt or solvate thereof is administered to a mammal conjointly with an imidazoline receptor agonist (e.g., an I₁, I₂, or I₃ receptor agonist) for the treatment of obesity, the mammal is in need of anti-psychotic treatment. In certain embodiments of methods of the invention wherein a compound of the present invention (e.g., (R)- or (S)-norfluoxetine) or a salt or solvate thereof is administered to a mammal conjointly with an imidazoline receptor agonist (e.g., an I₁, I₂, or I₃ receptor agonist) for the treatment of obesity, the mammal is being treated with one or more anti-psychotic agents.

In preferred embodiments of the present invention, the mammal being treated is a human.

Both fluoxetine and norfluoxetine exhibit functional activity versus the CB1 receptor. (S)-Fluoxetine is an inverse agonist of CB1, and (R)-fluoxetine is an antagonist of CB1. The racemate of norfluoxetine is an antagonist of CB1. Without wishing to be restricted by the proposal, this cannabinoid activity may mediate the utility of these compounds for the treatment of metabolic syndrome or the disorders associated with metabolic syndrome.

In certain embodiments of methods of the invention wherein a compound of the invention (e.g., (R)- or (S)-norfluoxetine or a salt or solvate thereof) is administered to a mammal being treated with one or more anti-psychotic agents, the anti-psychotic agents are selected from any suitable anti-psychotic agent. Suitable anti-psychotic agents include, but are not limited to, clozapine, olanzapine, quetiapine, risperidone, ziprasidone, aripiprazole, trifluoperazine, flupenthixol, loxapine, perphenazine, chlorpromazine, haloperidol, fluphenazine decanoate, thioridazine, or a pharmaceutically acceptable salt thereof.

In methods of the invention, wherein norfluoxetine enriched for the (R) or (S) enantiomer is administered conjointly with a calcium channel blocker, the calcium channel blocker may be chosen from any suitable calcium channel blocker. Calcium channel blockers suitable for said conjoint administration include, but are not limited to, those listed in Table 1 or their pharmaceutically acceptable salts.

TABLE 1

| | CAS NUMBERS FOR | |
|---|---|---|
| | SPECIFIC AND | |
| | REPRESENTATIVE | |
| COMPOUNDS | COMPOUNDS | REFERENCE |
| A-53930A | 182410-79-3 | JP 08208690, |
| | | Sankyo Co Ltd |
| AE-0047 | 133743-71-2 | Welfide Corp |
| AH-1058 | 228123-157 | Ajinomoto |
| AM-336 | | AMRAD Corporation |
| Amlodipine | 103129-82-4 | Novartis |
| AR-R18565 | | AstraZeneca |
| aranidipine | 86780-90-7 | Maruko Seiyaku |
| atosiban | 90779-69-4 | EP-00112809, |
| | | Ferring AB |
| azelnidipine | 123524-52-7 | EP 266922, |
| | | Sankyo |
| barnidipine | 104713-75-9 | DE-02904552, |
| | | Yamanouchi |
| BAY-Z-4406 | | Bayer |
| bepridil HCI | 68099-86-5 | Ortho-McNeil |
| bisaramil | 89194-77-4 | Richter Gedeon VG |
| buflomedil | 35543-24-9 | Laboratoire L |
| | | Lafon SA |
| CAI | 99519-84-3 | EP 304221, |
| | | NIH |
| CHF-1521 | | Chiesi Farmaceutici SpA |
| Cilnidipine | 133203-70-4 | Fujirebio KK |
| CP-060CP-060S | 183181-90-0 | WO 9500471, |
| | 183181-89-7 | Chugai |
| CPC-301 | | Questcor |
| CPC-317 | | Questcor |
| CPU-86017 | 149088-32-4 | EP 00538844, |
| | | China Pharmaceutical |
| | | University |
| Diltiazem HCl | 33286-22-5 | Hoechst Marion Roussel |
| Docosahexaenoic | 6217-54-5 | WO-09428891, |
| acid | | Martek Biosciences |
| Dotarizine | 84625-59-2 | EP 97340, |
| | | Ferrer Internacional SA |
| Efonidipine HCl | 111011-53-1 | US. Pat. No.-04843076, |
| | | Nissan Chemical |
| Elgodipine | 119413-55-7 | EP 302980, |
| | | (IQB) Instituto Invest y |
| | | Desarrolo Quimico-Biologico |
| | | SA |
| Fasudil | 103745-39-7 | EP 00187371, |
| | | Asahi Chemical Industry Co |
| | | Ltd. |
| FCE-28718 | | EP 0755931, |
| | | Pharmacia & UpjohnSpA |
| Felodipine | 72509-76-3 | AstraZeneca LP |
| FR-172516 | 188564-74-1 | Fujisawa Pharmaceutical Co |
| | | Ltd. |
| FRG-8701 | 108498-50-6 | Fujirebio KK |
| Furnidipine | 138661-03-7 | Cermol SA |
| Ipenoxazone | 104454-7 | Nippon Chemiphar Co Ltd. |
| Isradipine | 75695-93-1 | EP 00000150, |
| | | Novartis AG |
| JTV-519 | 145903-06-6 | WO 09212148, |
| | | Japan Tobacco Inc |
| kurtoxin-1 | | NIH |
| L-651582 | 79519-84-3 | EP 00151529, |
| | | Merck & Co Inc |
| lacidipine | 103890-78-4 | DE-03529997, |
| | | Glaxo Wellcome plc |
| lemildipine | 94739-29-4 | JP 59152373, |
| | | Merck & Co; Banyu |
| | | Pharmaceutical Co Ltd |
| lercanidipine | 100427-26-7 | EP-00153016, |
| | | Recordati SpA |
| LOE-908 | 149759-26-2 | Berlin Free |
| lomerizine | 101477-54-7 | EP-00159566, |
| | | Kanebo KK |
| LY-042826 | | Eli Lilly & Co |
| LY-393615 | | Eli Lilly & Co |
| manidipine | 89226-50-6 | EP-00094159, Takeda |
| | | Chemical Industries Ltd |
| NCC-1048 | | Eli Lilly & Co |
| nicardipine HCL | 54527-84-3 | Wyeth-Ayerst |
| nifedipine | 21829-25-4 | Bayer |
| nifelan | 21829-25-4 | Elan Corp |
| nilvadipine | 75530-68-6 | DE-02940833, Fujisawa |
| | | Pharmaceutical Co Ltd |
| nimodipine | 66085-59-4 | Bayer AG |
| NIP-142 | | WO 9804542, |
| | | Nissan Chemical |
| nisoldipine | 63675-72-9 | AstraZeneca |
| nitrendipine | 39562-70-4 | EP 0084054 |
| | | Vita- Invest SA |
| NS-7 | 178429-67-9 | WO 09067641, |
| | | Nippon Shinyaku |
| NW-1015 | 202825-46-5 | AU 711309 |
| | | Newron |
| Org-13061 | 198711-29-4 | Riom Laboratories |
| | | CERM SA |
| oxodipine | 90729-41-2 | Instituto Invet y Desarrollo |
| | | Quimico-Biologico SA |
| P-5 | | Universidad de Salamanca |
| PCA-50922 | 211307-87-8 | Alter SA |
| PCA-50938 | 152287-53-1 | Alter SA |
| PCA-50941 | 136941-85-0 | Alter SA |
| PD-029361 | | Pfizer Inc |
| PD-151307 | 247130-18-3 | Warner-Lambert |
| | 25925-12-2 | |
| PD-157667 | | Pfizer Inc |
| PD-158143 | | Pfizer Inc |
| PD-173212 | | WO 9854123, |
| | | Warner-Lambert |
| PD-176078 | 217170-95-1 | Elan Pharmaceuticals Inc |
| Pharmaprojects | | WO 9801121, |
| No 5898 | | Shionogi |
| Pharmaprojects | | NIH |
| No 6266 | | |
| Pharmaprojects | | NIH |
| No 6362 | | |
| Pharmaprojects | | Elan |
| No 6375 | | |
| Pharmaprojects | | Eisai |
| No 6429 | | |
| Plendil | 72509-76-3 | AstraZeneca |
| Pranidipine | 99522-79-9 | EP 173126, EP 145434, |
| | | Otsuka |
| QX-314 | 21306-56-9 | Roche Bioscience |
| R-verapamil | 38176-0202 | Celltech |
| | 38321-02-7 | |
| Ranolazine | 95635-55-5 | EP 126449, |
| | 95635-56-6 | Hoffmann-La Roche |
| RGH-2716 | 209264-08-4 | Richter Gedeon VG |
| S-312d | 120004-07-1 | JP 03052890, |
| | | Shionogi & Co. |
| SANK-71996 | | Sankyo Co Ltd |
| SB-201823A | 141429-64-3 | WO 09202502, |
| | | SmithKline Beecham |
| SB-237376 | | SmithKline Beecham plc |
| SD-3212 | 116476-17-6 | WO 08700838, Santen |
| | | Pharmaceutical Co Ltd. |
| Semotiadil | 116476-13-2 | JP 09012576, Santen |
| | | Pharmaceutical Co. Ltd. |
| SIB-1281 | | SIBIA Neurosciences Inc. |
| SKF-45675 | | SmithKline Beecham |
| | | Pharmaceutical (US) |
| SKT-M26 | | Toyama Medical and |
| | | Pharmaceutical University |
| SL-34,0829-08 | | Sanofi-Synthelabo |
| SNX-185 | 149797-45-5 | Elan |
| SNX-239 | 162995-02-0 | Elan |
| SUN-N5030 | | Suntory Institute for |
| | | Biomedical Research |
| T-477 | 136929-56-1 | EP 00441539, |
| | | Tanabe Seiyaku Co Ltd |
| TA-993 | 122024-98-0 | Tanabe Seiyaku Co Ltd |
| Tamolarizine | 128229-52-7 | EP 00354068, |
| | | Nippom Chemiphar Co Ltd |
| Teczem | 120784-30-7 | Aventis Pharma |
| Temiverine HCl | 129927-33-9 | Nippon Shinyaku |
| Tenosal | 95232-68-1 | EP 123094, |
| | | Medea Research |
| Terodiline | 15793-40-5 | Pharmacia & Upjohn AB |
| TH-1177 | 266001-66-5 | University of Virginia |
| TH-9229 | | WO 09607415, |
| | | Theratechnologies Inc. |
| U-92032 | 142223-92-5 | WO 09204338, |
| | | Pharmacia & Upjohn Co |
| vatanidipine | 133743-71-2 | EP 257616, |
| HCl | 116308-55-5 | Welfide Corporation |
| verapamil HCI | 52-53-9 | Searle |
| Verelan | | Elan Corp plc |
| vexibinol | 97938-30-2 | Kuraray Co Ltd |
| vintoperol | 106498-99-1 | U.S. Pat. No. 4806545, |
| | | Takata Seiyaku |
| YM-430 | 153192-22-4 | Yamanouchi Pharm Co Ltd |
| ziconotide | 107452-89-1 | WO 09107980, |
| | | Elan Pharmaceuticals Inc |

Other calcium channel blockers suitable for use in methods of the invention include, but are not limited to, benidipine, darodipine, lotrel, lidoflazine, mepirodipine, niguldipine, niludipine, nivaldipine, perhexyline, ryosidine, anipamil, fendiline, flunarizine, gallopamil, mibefradil, prenylamine, or tiapamil or their pharmaceutically acceptable salts.

In certain embodiments, wherein norfluoxetine enriched for the (R) or (S) enantiomer is administered conjointly with a calcium channel blocker for methods of the invention, the calcium channel blocker is amlodipine or a pharmaceutically acceptable salt thereof. In certain embodiments, wherein norfluoxetine enriched for the (R) or (S) enantiomer is administered conjointly with a calcium channel blocker for methods of the invention, the calcium channel blocker is amlodipine enriched for the (R) or (S) enantiomer or a pharmaceutically acceptable salt thereof. An enantiomerically enriched mixture may comprise, for example, at least 60 mol percent of one enantiomer, or more preferably at least 75, 90, 95, or even 99 mol percent. In certain embodiments, amlodipine is enriched in the (R) enantiomer. In certain embodiments, (R)-amlodipine is substantially free of the (S)-enantiomer. In certain embodiments, amlodipine is enriched in the (S) enantiomer. In certain embodiments, (S)-amlodipine is substantially free of the (R)-enantiomer.

In methods of the invention, wherein norfluoxetine enriched for the (R) or (S) enantiomer is administered conjointly with a statin, the statin may be chosen from any statin known in the art. Statins suitable for said conjoint administration include, but are not limited to, mevastatin ((2S)-2-methyl butanoic acid (1S,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthalenyl ester), atorvastatin ((βR,δR)-2-(4-fluorophenyl)-β,δ-dihydroxy-5-(1-methylethyl)-3-phenyl-4-[(phenylamino)carbonyl]-1H-Pyrrole-1-heptanoic acid), fluvastatin ((3R,5 S,6E)-rel-7-[3-(4-fluorophenyl)-1-(1-methylethyl)-1H-indol-2-yl]-3,5-dihydroxy-6-heptenoic acid), lovastatin (2(S)-2-methyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), pravastatin ((βR,δR,1S,2S,6S,8S,8aR)-1,2,6,7,8,8a-hexahydro-β,β,6-trihydroxy-2-methyl-8-[(2S)-2-methyl-1-oxobutoxy]-1-naphthaleneheptanoic acid), simvastatin (2,2-dimethyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), rosuvastatin ((3R,5 S,6E)-7-[4-(4-fluorophenyl)-6-(1-methylethyl)-2-[methyl(methylsulfonyl)amino]-5-pyrimidinyl]-3,5-dihydroxy-6-heptenoic acid), eptastatin, pitavastatin ((3R,5S,6E)-7-[2-cyclopropyl-4-(4-fluorophenyl)-3-quinolinyl]-3,5-dihydroxy-6-heptenoic acid), cerivastatin ((3R,5S,6E)-7-[4-(4-fluorophenyl)-5-(methoxymethyl)-2,6-bis(1-methylethyl)-3-pyridinyl]-3,5-dihydroxy-6-heptenoic acid), berivastatin ((R*,S*-(E)-7-(4-(4-fluorophenyl)spiro(2H-1-benzopyran-2,1′-cyclopentan)-3-yl)-3,5-dihydroxy-ethyl ester), dalvastatin ((4R,6S)-rel-6-[(1E)-2-[2-(4-fluoro-3-methylphenyl)-4,4,6,6-tetramethyl-1-cyclohexen-1-yl]ethenyl]tetrahydro-4-hydroxy-, 2H-Pyran-2-one), glenvastatin ((4R,6S)-6-[(1E)-2-[4-(4-fluorophenyl)-2-(1-methylethyl)-6-phenyl-3-pyridinyl]ethenyl]tetrahydro-4-hydroxy-2H-Pyran-2-one), RP 61969 ([2S-[2a(E),4β]]-; 4-(4-fluorophenyl)-2-(1-methylethyl)-3-[2-(tetrahydro-4-hydroxy-6-oxo-2H-pyran-2-yl)ethenyl]-1 (2H)-isoquinolinone), SDZ-265859, BMS-180431 ((3R,5S,6E)-rel-9,9-bis(4-fluorophenyl)-3,5-dihydroxy-8-(1-methyl-1H-tetrazol-5-yl)-6,8-Nonadienoic acid), CP-83101 ((3R,5 S,6E)-rel-3,5-dihydroxy-9,9-diphenyl-6,8-Nonadienoic acid methyl ester), dihydromevinolin ((2S)-2-methyl-butanoic acid (1S,3S,4aR,7S,8S,8aS)-1,2,3,4,4a,7,8,8a-octahydro-3,7-dimethyl-8-[2-[(2R,4R)-tetrahydro-4-hydroxy-6-oxo-2H-pyran-2-yl]ethyl]-1-naphthalenyl ester), and L-669262 (2,2-dimethyl-butanoic acid (1S,7R,8R,8aR)-1,2,6,7,8,8a-hexahydro-3,7-dimethyl-6-oxo-8-[2-[(2R,4R)-tetrahydro-4-hydroxy-6-oxo-2H-pyran-2-yl]ethyl]-1-naphthalenyl ester), or their pharmaceutically acceptable salts.

For example, statins suitable for use in the methods of this invention include statins of formula 1:

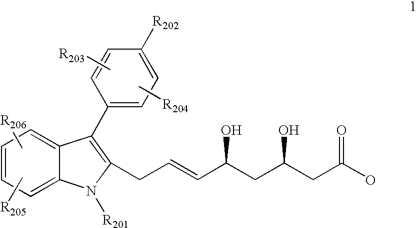

wherein
R₂₀₁ is selected from alkyl, alkenyl, alkynyl, cycloalkyl or aralkyl;
R₂₀₂, R₂₀₃ and R₂₀₄ are independently selected from hydrogen, halogen, alkyl, alkenyl or alkynyl; and
R₂₀₅ and R₂₀₆ are independently selected from hydrogen, halogen, alkyl, alkenyl, alkynyl, cycloalkyl, aralkyl, alkoxy or aralkoxy;
or enantiomers or salts or hydrates thereof.

Other statins suitable for use in the methods of this invention include statins of formula 2:

A-B

wherein

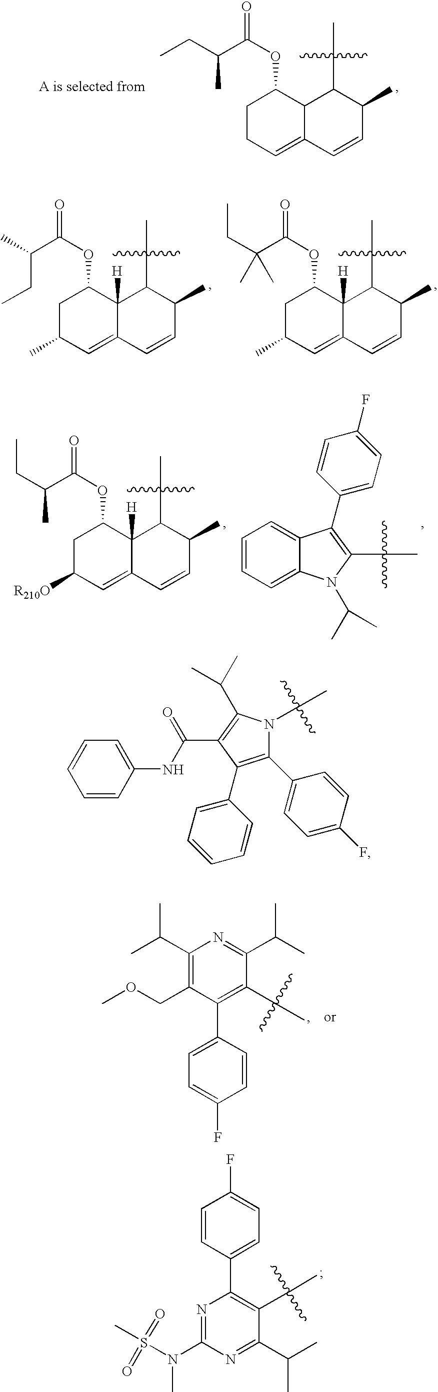

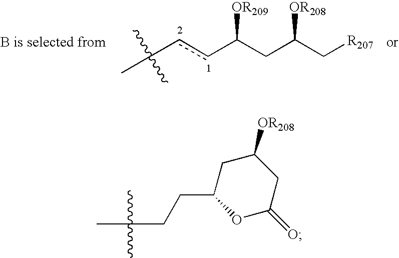

C1 and C2 are joined by a single or a double bond;
R₂₀₇ is selected from CO₂R₂₁₅, CONR₂₁₁R₂₁₂ or CH₂OR₂₁₃, or R₂₀₇ and R₂₀₉ can form a lactone;
R₂₁₅ is selected from H or a cationic salt moiety, or CO₂R₂₁₅ forms a pharmaceutically acceptable ester moiety;
R₂₀₈, R₂₀₉ and R₂₁₀ are independently selected from H, C(O)R₂₁₄ or C(O)NR₂₁₁R₂₁₂;
R₂₁₁ and R₂₁₂ are independently selected from H, alkyl, alkenyl or alkynyl;
R₂₁₃ is selected from H or C(O)R₂₁₄; and
R₂₁₄ is selected from alkyl, alkenyl or alkynyl.

Other statins suitable for use in the methods of this invention include statins of formula 3:

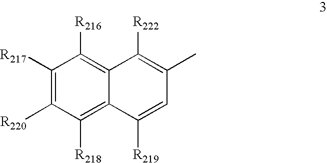

wherein
R₂₂₂ is selected from

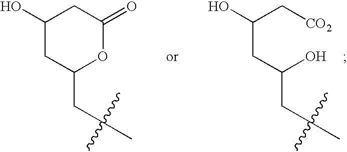

R₂₁₆ is selected from OH, C₆H₅CO₂ or R₂₂₁CO₂;
R₂₂₁ is a branched or straight C₁-C₅ alkyl, C₂-C₅ alkenyl, or C₂-C₅ alkynyl;
R₂₁₇, R₂₁₈ and R₂₁₉ are independently selected from H, C₁-C₅ alkyl, C₂-C₅ alkenyl, C₂-C₅ alkynyl or C₁-C₅ acyl; and
R₂₂₀ is selected from H or CH₃.

Other statins suitable for use in the methods of this invention include statins of formula 4:

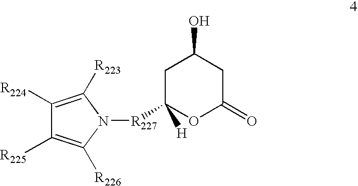

wherein
R₂₂₇ is —CH₂—, —CH₂—CH₂—, —CH₂—CH₂—CH₂— or —CH₂—CH(CH₃)—.;
R₂₂₃ is 1-naphthyl; 2-naphthyl; cyclohexyl; norbornenyl; 2-, 3-, or 4-pyridinyl; phenyl, phenyl substituted with fluorine, chlorine, bromine, hydroxyl; trifluoromethyl; alkyl, alkenyl, or alkynyl of from one to four carbon atoms, alkoxy of from one to four carbon atoms, or alkanoyloxy of from two to eight carbon atoms;
Either R₂₂₄ or R₂₂₅ is —CONR₂₂₈R₂₂₉ where R₂₂₈ and R₂₂₉ are independently hydrogen; alkyl, alkenyl, or alkynyl of from one to six carbon atoms; 2-, 3-, or 4-pyridinyl; phenyl; phenyl substituted with fluorine, chlorine, bromine, cyano, trifluoromethyl, or carboalkoxy of from three to eight carbon atoms; and the other of R₂₂₄ or R₂₂₅ is hydrogen; alkyl, alkenyl, or alkynyl of from one to six carbon atoms; cyclopropyl; cyclobutyl; cyclopentyl; cyclohexyl; phenyl; or phenyl substituted with fluorine, chlorine, bromine, hydroxyl; trifluoromethyl; alkyl, alkenyl, or alkynyl of from one to four carbon atoms, alkoxy of from one to four carbon atoms, or alkanoyloxy of from two to eight carbon atoms; and
R₂₂₆ is alkyl, alkenyl, or alkynyl of from one to six carbon atoms; cyclopropyl; cyclobutyl; cyclopentyl; cyclohexyl; or trifluoromethyl;
or the hydroxyl acids, and pharmaceutically acceptable salts thereof, derived from the opening of the lactone ring.

Other statins suitable for use in the methods of this invention include statins of formula 5:

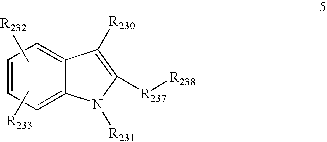

wherein
one of R₂₃₀ and R₂₃₁ is

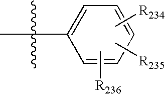

and the other is primary or secondary C₁₋₆ alkyl, alkenyl, or alkynyl not containing an asymmetric carbon atom, C₃₋₆ cycloalkyl or phenyl-(CH₂)ₘ—;
R₂₃₄ is selected from hydrogen, C₁₋₃ alkyl, C₂-C₄ alkenyl, C₂-C₄ alkynyl, n-butyl, i-butyl, t-butyl, C₁₋₃ alkoxy, n-butoxy, i-butoxy, trifluoromethyl, fluoro, chloro, phenoxy or benzyloxy;
R₂₃₅ is selected from hydrogen, C₁₋₃ alkyl, C₂-C₃ alkenyl, C₂-C₃ alkynyl, C₁₋₃ alkoxy, trifluoromethyl, fluoro, chloro, phenoxy or benzyloxy;
R₂₃₆ is selected from hydrogen, C₁₋₂ alkyl, C₂ alkenyl, C₂ alkynyl, C₁₋₂ alkoxy, fluoro or chloro;
m is selected from 1, 2 or 3, with the provisos that both R₂₃₅ and R₂₃₆ must be hydrogen when R₂₃₄ is hydrogen, R₂₃₆ must be hydrogen when R₂₃₅ is hydrogen, not more than one of R₂₃₄ and R₂₃₅ is trifluoromethyl, not more than one of R₂₃₄ and R₂₃₅ is phenoxy, and not more than one of R₂₃₄ and R₂₃₅ is benzyloxy;
R₂₃₂ is selected from hydrogen, C₁₋₃ alkyl, C₂-C₄ alkenyl, C₂-C₄ alkynyl, n-butyl, i-butyl, t-butyl, C₃₋₆ cycloalkyl, C₁₋₃ alkoxy, n-butoxy, i-butoxy, trifluoromethyl, fluoro, chloro, phenoxy or benzyloxy;
R₂₃₃ is selected from hydrogen, C₁₋₃ alkyl, C₂-C₃ alkenyl, C₂-C₃ alkynyl, C₁₋₃ alkoxy, trifluoromethyl, fluoro, chloro, phenoxy or benzyloxy, with the provisos that R₂₃₃ must be hydrogen when R₂₃₂ is hydrogen, not more than one of R₂₃₂ and R₂₃₃ is trifluoromethyl, not more than one of R₂₃₂ and R₂₃₃ is phenoxy, and not more than one of R₂₃₂ and R₂₃₃ is benzyloxy;
R₂₃₇ is selected from —(CH₂)ₙ— or —CH═CH—, wherein n is 0, 1, 2 or 3;
R₂₃₈ is selected from

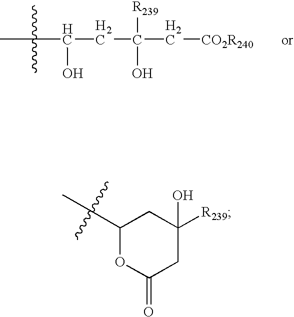

R₂₃₉ is selected from hydrogen, or C₁₋₃ alkyl, C₂-C₃ alkenyl, or C₂-C₃ alkynyl;
R₂₄₀ is selected from hydrogen, R₂₄₁ or M;
R₂₄₁ is a physiologically acceptable and hydrolyzable ester group; and
M is a pharmaceutically acceptable cation.

Other statins suitable for use in the methods of this invention include statins of formula 6:

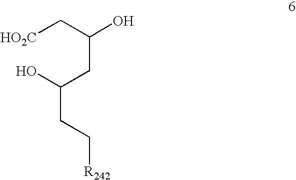

wherein
R₂₄₂ is selected from

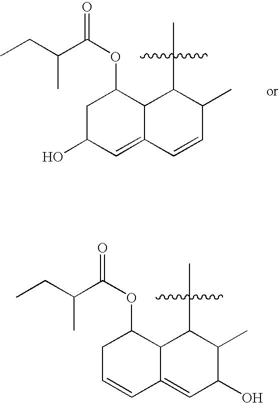

or ring-closed lactones, salts or esters thereof.

Other statins suitable for use in the methods of this invention include statins of formula 7:

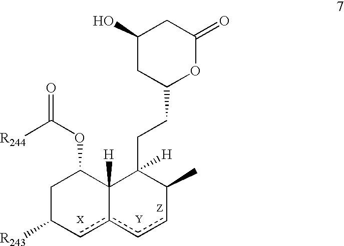

wherein
R₂₄₃ is selected from H or CH₃;
R₂₄₄ is selected from 1,1-dimethylpropyl; C₃₋₁₀cycloalkyl; C₂₋₁₀alkenyl; C₁₋₁₀CF₃-substituted alkyl; phenyl; halophenyl; phenyl-C₁₋₃alkyl; substituted phenyl-C₁₋₃alkyl in which the substituent is halo, C₁₋₃alkyl or C₁₋₃alkoxy;
the dotted lines at X, Y and Z represent possible double bonds, said double bonds, when any are present, being either X and Z in combination or X, Y or Z alone;
or the corresponding dihydroxy acid of formula 206a

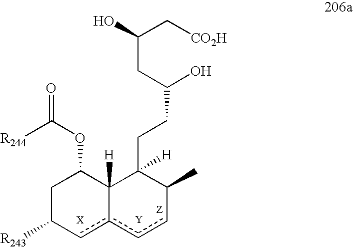

or a pharmaceutically acceptable salt of said acid, a C₁₋₄alkyl ester of said acid or a phenyldimethylamino-, or acetylamino-substituted-C₁₋₄alkyl ester of said acid.

Other statins suitable for use in the methods of this invention include statins of formula 8:

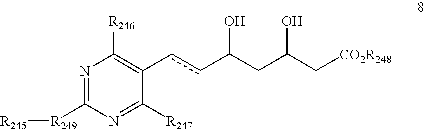

wherein
R₂₄₅ is lower alkyl, alkenyl, alkynyl, aryl or aralkyl, each of which may have one or more substituents;
R₂₄₆ and R₂₄₇ independently are selected from hydrogen, lower alkyl, alkenyl, alkynyl, or aryl, and each of said lower alkyl, alkenyl, alkynyl and aryl may have one or more substituents;
R₂₄₈ is hydrogen, lower alkyl, alkenyl, alkynyl, or a cation capable of forming a non-toxic pharmaceutically acceptable salt;
R₂₄₉ is sulfur, oxygen, or sulfonyl, or imino which may have a substituent; and
the dotted line represents the presence or absence of a double bond;
or the corresponding ring-closed lactone.

In certain embodiments, wherein norfluoxetine enriched for the (R) or (S) enantiomer is administered conjointly with a statin for methods of the invention, the statin is chosen from atorvastatin or simvastatin, or a pharmaceutically acceptable salt thereof. In certain embodiments, wherein norfluoxetine enriched for the (R) or (S) enantiomer is administered conjointly with a statin for methods of the invention, the statin is atorvastatin or a pharmaceutically acceptable salt thereof. In certain embodiments, wherein norfluoxetine enriched for the (R) or (S) enantiomer is administered conjointly with a statin for methods of the invention, the statin is simvastatin or a pharmaceutically acceptable salt thereof.

The synthesis of each of the statins set forth above can be achieved by methods well-known in the art. The synthesis of various statins is set forth in U.S. RE37314 E, U.S. Pat. No. 4,444,784, U.S. Pat. No. 4,346,227, U.S. Pat. No. 5,354,772, U.S. Pat. No. 4,681,893 and US 2005/0228042.

In methods of the invention, wherein norfluoxetine enriched for the (R) or (S) enantiomer is administered conjointly with an imidazoline receptor agonist (e.g., an I₁, I₂, or I₃ receptor agonist), the imidazoline receptor agonist (e.g., an I₁, I₂, or I₃ receptor agonist) may be chosen from any suitable imidazoline receptor agonist (e.g., an I₁, I₂, or I₃ receptor agonist). Imidazoline receptor agonists (e.g., an I₁, I₂, or I₃ receptor agonists) suitable for said conjoint administration include, but are not limited to clonidine, benazoline, rilmenidine, idazoxan, RS-45041-190, 2-BFI, BU 224, BU 239, LSL 60101, RX 821029, or pharmaceutically acceptable salts thereof. In certain embodiments, the imidazoline receptor agonist is clonidine or a pharmaceutically acceptable salt thereof. In certain embodiments, the imidazoline receptor agonist is idazoxan or a pharmaceutically acceptable salt thereof. In certain embodiments of methods of the invention wherein norfluoxetine enriched for the (R) enantiomer is administered conjointly with an imidazoline receptor agonist, the imidazoline receptor agonist is not moxonidine. In certain embodiments of methods of the invention wherein norfluoxetine enriched for the (R) or (S) enantiomer is administered conjointly with an imidazoline receptor agonist, the imidazoline receptor agonist is not moxonidine.

As used herein, the term “obesity” includes both excess body weight and excess adipose tissue mass in an animal. An obese individual is one having a body mass index of ≧30 kg/m². While the animal is typically a human, the invention also encompasses the treatment of non-human mammals. The treatment of obesity, as provided in methods of the present invention, contemplates not only the treatment of individuals who are defined as “obese”, but also the treatment of individuals with weight gain that if left untreated may lead to the development of obesity.

The term “hydrate” as used herein, refers to a compound formed by the union of water with the parent compound.

The term “metabolite” is intended to encompass compounds that are produced by metabolism of the parent compound under normal physiological conditions. For example, an N-methyl group may be cleaved to produce the corresponding N-desmethyl metabolite. Preferred metabolites of the present invention include those that exhibit similar activity to their parent compound (e.g., metabolites that are suitable for the treatment of metabolic syndrome or a disorder associated with metabolic syndrome).

The term “solvate” as used herein, refers to a compound formed by solvation (e.g., a compound formed by the combination of solvent molecules with molecules or ions of the solute).

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

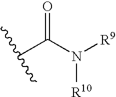

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

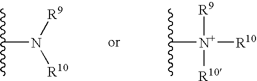

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

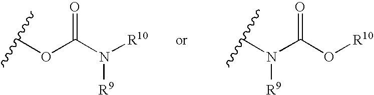

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

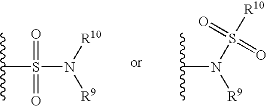

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R⁹, wherein R⁹ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R⁹, wherein R⁹ represents a hydrocarbyl.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

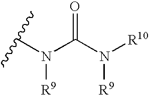

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Methods of preparing substantially isomerically pure compounds are known in the art. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longman Scientific and Technical Ltd., Essex, 1991, pp. 809-816; and Heller, Acc. Chem. Res. 23: 128 (1990).

The amount of active agent(s) (e.g., norfluoxetine, e.g., enriched for (R)- or (S)-norfluoxetine) administered can vary with the patient, the route of administration and the result sought. Optimum dosing regimens for particular patients can be readily determined by one skilled in the art. In general, a therapeutically relevant dose for the treatment or prevention of metabolic syndrome is less than the dose required to obtain a therapeutically relevant dose for the treatment of major depressive disorder or obsessive compulsive disorder.

(R)- or (S)-Norfluoxetine may be administered to an individual in need thereof. In certain embodiments, the individual is a mammal such as a human, or a non-human mammal. When administered to an individual, the norfluoxetine and/or another active agent can be administered as a pharmaceutical composition containing, for example, the agent or agents and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery system, e.g., a skin patch.

The term “low enough pyrogen activity”, with reference to a pharmaceutical preparation, refers to a preparation that does not contain a pyrogen in an amount that would lead to an adverse effect (e.g., irritation, fever, inflammation, diarrhea, respiratory distress, endotoxic shock, etc.) in a subject to which the preparation has been administered. For example, the term is meant to encompass preparations that are free of, or substantially free of, an endotoxin such as, for example, a lipopolysaccharide (LPS).

A pharmaceutically acceptable carrier can contain physiologically acceptable agents that act, for example, to stabilize or to increase the absorption of a compound such as norfluoxetine. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, an active agent. Liposomes, for example, which consist of phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) containing norfluoxetine can be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments norfluoxetine may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. The most preferred route of administration is the oral route.

The formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the active agents include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal, vaginal, or urethral administration may be presented as a suppository, which may be prepared by mixing one or more active agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions of this invention suitable for parenteral administration comprise one or more active agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of active agents to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular active agent employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the agents employed in the pharmaceutical composition at levels lower than that required in order to achieve the desired therapeutic effect and gradually increase the dosage until the desired effect is achieved.

In general, a suitable daily dose of an active agent will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, the compound (e.g., norfluoxetine) of the present invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

It is contemplated that the compound (e.g., norfluoxetine enriched for the (R) or (S) enantiomer) of the present invention will be administered to a subject (e.g., a mammal, preferably a human) in a therapeutically effective amount (dose). By “therapeutically effective amount” is meant the concentration of a compound that is sufficient to elicit the desired therapeutic effect (e.g., treatment or prevention of metabolic syndrome, or the specific disorders associated with metabolic syndrome). It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with an active agent. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

It is contemplated that a therapeutically effective amount (dose) of the compound (e.g., norfluoxetine enriched for the (R) or (S) enantiomer) to be administered to a subject (e.g., a mammal, preferably a human) will be in the range of 1 mg/day and 100 mg/day. In certain preferred embodiments, the therapeutically effective amount of the compound to be administered to a subject will be in a range of 1 mg/day and 60 mg/day. In certain embodiments, the therapeutically effective amount of the compound to be administered to a subject will be in a range of 1 mg/day and 40 mg/day. In certain embodiments, the therapeutically effective amount of the compound to be administered to a subject will be in a range of 1 mg/day and 10 mg/day.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention may contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs form the various salt forms in certain physical properties, such as solubility in polar solvents, but otherwise the salts are equivalent to the parent form of the compound for the purposes of the present invention.

As a particular example, this invention includes the pharmaceutically acceptable acid addition salts of norfluoxetine, such as (R)- or (S)-norfluoxetine. Since norfluoxetine is an amine, it is basic in nature and accordingly reacts with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. Preferred pharmaceutically acceptable acid addition salts include those formed with mineral acids such as hydrochloric acid and hydrobromic acid, and those formed with organic acids such as fumaric acid, tartaric acid and maleic acid. In certain embodiments, the tartaric acid is (D)-tartaric acid and the resulting salt is the (D)-tartrate salt. In certain embodiments, the pharmaceutically acceptable salt is (R)-norfluoxetine (D)-tartrate.

The pharmaceutically acceptable acid addition salts of norfluoxetine can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Norfluoxetine can be prepared by any of a number of methods generally known in the art. For example, there are several methods provided in the literature for making the racemate of norfluoxetine (U.S. Pat. No. 4,313,896). The racemate of norfluoxetine in turn can be resolved, if desired, into its (S) and (R) components by standard methods. In particular, norfluoxetine can be reacted with an enantiomerically pure chiral derivatizing agent, resolved on the basis of the different physicochemical properties of the diastereomeric derivatives, and then converted to the two separate enantiomers of norfluoxetine. One particularly preferred method of accomplishing this derivatization is analogous to that described in Robertson et al., J. Med. Chem., 31, 1412 (1988), wherein fluoxetine was reacted with an optically active form of 1-(1-naphthyl)ethyl isocyanate to form a urea derivative of fluoxetine. A similar mixture of norfluoxetine diastereomeric ureas can be separated through high pressure liquid chromatography into the individual diastereomers. Each individual diastereomer, in turn, can then be hydrolyzed to the individual enantiomers of norfluoxetine.

The pharmaceutically acceptable acid addition salts are typically formed by reacting norfluoxetine with an equimolar or excess amount of acid. The reactants are generally combined in a mutual solvent such as diethyl ether or benzene, and the salt normally precipitates out of solution within about one minute to 10 days, and can be isolated by filtration.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The present invention provides a kit comprising:
a) a first pharmaceutical formulation comprising a compound of the present invention (e.g., norfluoxetine enriched for the (R) or the (S) enantiomer);
b) a second pharmaceutical formulation comprising at least one of the following: a statin, a calcium channel blocker, halofenate, captopril, or an imidazoline receptor agonist (e.g., an I₁, I₂, or I₃ receptor agonist); and
c) instructions for the administration of the first and second pharmaceutical formulations.

The present invention provides a kit comprising:
a) a first pharmaceutical formulation comprising a compound of the present invention (e.g., norfluoxetine enriched for the (R) or the (S) enantiomer);
b) a second pharmaceutical formulation comprising at least one of the following: atorvastatin or simvastatin; and
c) instructions for the administration of the first and second pharmaceutical formulations.

The present invention provides a kit comprising:
a) a first pharmaceutical formulation comprising a compound of the present invention (e.g., norfluoxetine enriched for the (R) or the (S) enantiomer);
b) a second pharmaceutical formulation comprising amlodipine; and
c) instructions for the administration of the first and second pharmaceutical formulations.

The present invention provides a kit comprising:
a) a first pharmaceutical formulation comprising a compound of the present invention (e.g., norfluoxetine enriched for the (R) or the (S) enantiomer);
b) a second pharmaceutical formulation comprising halofenate; and
c) instructions for the administration of the first and second pharmaceutical formulations.

The present invention provides a kit comprising:
a) a first pharmaceutical formulation comprising a compound of the present invention (e.g., norfluoxetine enriched for the (R) or the (S) enantiomer);
b) a second pharmaceutical formulation comprising captopril; and
c) instructions for the administration of the first and second pharmaceutical formulations.

The present invention provides a kit comprising:
a) a first pharmaceutical formulation comprising a compound of the present invention (e.g., norfluoxetine enriched for the (R) or the (S) enantiomer);
b) a second pharmaceutical formulation comprising clonidine; and
c) instructions for the administration of the first and second pharmaceutical formulations.

The present invention provides a kit comprising:
a) a first pharmaceutical formulation comprising a compound of the present invention (e.g., norfluoxetine enriched for the (R) or the (S) enantiomer);
b) a second pharmaceutical formulation comprising idazoxan; and
c) instructions for the administration of the first and second pharmaceutical formulations.

In certain embodiments, the invention relates to a method for conducting a pharmaceutical business, by manufacturing a formulation of norfluoxetine enriched for the (R) or (S) enantiomer to be administered conjointly with a statin, a calcium channel blocker, halofenate, captopril, or an imidazoline receptor agonist (e.g., an I₁, I₂, or I₃ receptor agonist), or a kit as described herein, and marketing to healthcare providers the benefits of using the formulation or kit in the treatment or prevention of metabolic syndrome or the specific disorders associated with metabolic syndrome.

In certain embodiments, the invention relates to a method for conducting a pharmaceutical business, by providing a distribution network for selling a formulation of norfluoxetine enriched for the (R) or (S) enantiomer to be administered conjointly with a statin, a calcium channel blocker, halofenate, captopril, or an imidazoline receptor agonist (e.g., an I₁, I₂, or I₃ receptor agonist), or kit as described herein, and providing instruction material to patients or physicians for using the formulation to treat or prevent metabolic syndrome or the specific disorders associated with metabolic syndrome.

In certain embodiments, the invention comprises a method for conducting a pharmaceutical business, by determining an appropriate formulation and dosage of norfluoxetine enriched for the (R) or (S) enantiomer to be administered conjointly with a statin, a calcium channel blocker, halofenate, captopril, or an imidazoline receptor agonist (e.g., an I₁, I₂, or I₃ receptor agonist) in the treatment or prevention of metabolic syndrome or the specific disorders associated with metabolic syndrome, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of norfluoxetine enriched for the (R) or (S) enantiomer to be administered conjointly with a statin, a calcium channel blocker, halofenate, captopril, or an imidazoline receptor agonist (e.g., an I₁, I₂, or I₃ receptor agonist) in the treatment or prevention of metabolic syndrome or the specific disorders associated with metabolic syndrome, and licensing, to a third party, the rights for further development and sale of the formulation.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing metabolic syndrome or a disorder associated with metabolic syndrome in a mammal, comprising administering norfluoxetine enriched for the (R) or (S) enantiomer conjointly with halofenate, captopril, a statin, a calcium channel blocker, an imidazoline receptor agonist, or a pharmaceutically acceptable salt thereof.

2. The method of claim 1, characterized by one or more of the following:
the disorder associated with metabolic syndrome is obesity, diabetes, hypertension, or hyperlipidemia;
the statin is atorvastatin, simvastatin, or a pharmaceutically acceptable salt thereof;
the calcium channel blocker is amlodipine or a pharmaceutically acceptable salt thereof;
the calcium channel blocker is amlodipine or a pharmaceutically acceptable salt thereof enriched for the (R) or (S) enantiomer;
the halofenate or a pharmaceutically acceptable salt thereof is enriched for the (R) or (S) enantiomer;
the imidazoline receptor agonist is clonidine, idazoxan, or a pharmaceutically acceptable salt thereof;
said mammal is a human; or
the norfluoxetine enriched for the (R) or (S) enantiomer is provided as a salt of norfluoxetine enriched for the (R) or (S) enantiomer or a solvate thereof.

3. A kit comprising:
a) a first pharmaceutical formulation comprising norfluoxetine enriched for the (R) or (S) enantiomer;
b) a second pharmaceutical formulation comprising at least one of the following: a statin, a calcium channel blocker, halofenate, captopril, an imidazoline receptor agonist, or a pharmaceutically acceptable salt thereof; and
c) instructions for the administration of the first and second pharmaceutical formulations.

4. The kit of claim 3, characterized by one or more of the following:
the statin is atorvastatin or a pharmaceutically acceptable salt thereof;
the statin is simvastatin or a pharmaceutically acceptable salt thereof;
the calcium channel blocker is amlodipine or a pharmaceutically acceptable salt thereof;
the calcium channel blocker is amlodipine enriched for the (R) or (S) enantiomer or a pharmaceutically acceptable salt thereof;
the halofenate is enriched for the (R) or (S) enantiomer or a pharmaceutically acceptable salt thereof;
the imidazoline receptor agonist is clonidine or a pharmaceutically acceptable salt thereof;
the imidazoline receptor agonist is idazoxan or a pharmaceutically acceptable salt thereof; or
the norfluoxetine enriched for the (R) or (S) enantiomer is provided as a salt of norfluoxetine enriched for the (R) or (S) enantiomer or a solvate thereof.

5. A method for conducting a pharmaceutical business, comprising:
a. manufacturing a formulation of norfluoxetine enriched for the (R) or (S) enantiomer to be administered conjointly with a statin, a calcium channel blocker, halofenate, captopril, an imidazoline receptor agonist, or a pharmaceutically acceptable salt thereof, or a kit of claim 3; and
b. marketing to healthcare providers the benefits of using the formulation or kit in the treatment or prevention of metabolic syndrome or a disorder associated with metabolic syndrome.

6. A method for conducting a pharmaceutical business, comprising:
a. providing a distribution network for selling the formulation or kit as described in claim 5; and
b. providing instruction material to patients or physicians for using the formulation or kit to treat or prevent metabolic syndrome or a disorder associated with metabolic syndrome.

7. A method for conducting a pharmaceutical business, comprising:
a. determining an appropriate formulation and dosage of norfluoxetine enriched for the (R) or (S) enantiomer to be administered conjointly with a statin, a calcium channel blocker, halofenate, captopril, an imidazoline receptor agonist, or a pharmaceutically acceptable salt thereof in the treatment or prevention of metabolic syndrome or disorder associated with metabolic syndrome;
b. conducting therapeutic profiling of formulations identified in step (a), for efficacy and toxicity in animals; and
c. providing a distribution network for selling a preparation identified in step (b) as having an acceptable therapeutic profile; and
d. optionally including an additional step of providing a sales group for marketing the preparation to healthcare providers.

8. A method for conducting a pharmaceutical business, comprising:
a. determining an appropriate formulation and dosage of norfluoxetine enriched for the (R) or (S) enantiomer to be administered conjointly with a statin, a calcium channel blocker, halofenate, captopril, an imidazoline receptor agonist, or a pharmaceutically acceptable salt thereof in the treatment or prevention of metabolic syndrome or a disorder associated with metabolic syndrome; and
b. licensing, to a third party, the rights for further development and sale of the formulation.

9. The method of any one of claims 5-8, characterized by one or more of the following:
the disorder associated with metabolic syndrome comprise obesity, diabetes, hypertension, or hyperlipidemia;
the statin is atorvastatin or a pharmaceutically acceptable salt thereof;
the statin is simvastatin or a pharmaceutically acceptable salt thereof;
the calcium channel blocker is amlodipine or a pharmaceutically acceptable salt thereof;
the calcium channel blocker is amlodipine enriched for the (R) or (S) enantiomer or a pharmaceutically acceptable salt thereof;
the halofenate is enriched for the (R) or (S) enantiomer or a pharmaceutically acceptable salt thereof;
the imidazoline receptor agonist is clonidine or a pharmaceutically acceptable salt thereof;
the imidazoline receptor agonist is idazoxan or a pharmaceutically acceptable salt thereof; or
the norfluoxetine enriched for the (R) or (S) enantiomer is provided as a salt of norfluoxetine enriched for the (R) or (S) enantiomer or a solvate thereof.
